SEQUENCE LISTING (6 sequences)

SEQ ID NO: 1 - protein, 409 aa, Arabidopsis thaliana
MASISRILPTDGRLSQCRINTSWVPSTTRTQTHLDFPKLVSVSNSGISLRIQNSKPISPVFALEATSSRRVVCKAAAGVSGDLPESTPKELSQYEKIIELLTTLFPLWVILGTLVGIFKPSLVTWLETDLFTLGLGFLMLSMGLTLTFEDFRRCLRNPWTVGVGFLAQYMIKPILGFLIAMTLKLSAPLATGLILVSCCPGGQASNVATYISKGNVALSVLMTTCSTIGAIIMTPLLTKLLAGQLVPVDAAGLALSTFQVVLVPTIIGVLANEFFPKFTSKIITVTPLIGVILTTLLCASPIGQVADVLKTQGAQLILPVALLHAAAFAIGYWISKFSFGESTSRTISIECGMQSSALGFLLAQKHFTNPLVAVPSAVSVVCMALGGSGLAVFWRNLPIPADDKDDFKE

SEQ ID NO: 2 - DNA, 1230 bp, Arabidopsis thaliana
atggcttccatttccagaatcttaccaacagatggcagattaagtcaatgcagaatcaacacctcgtgggttccttctacaaccagaactcaaactcacttagattttcccaagttagtttctgttagtaactctgggataagtttgaggattcagaatagtaaacctattagccctgtctttgctcttgaagcaacttcctccaggagggttgtttgcaaagctgctgctggtgtgtcaggtgacttacctgagagtactcctaaggaacttagtcagtatgagaagattattgagcttttgacaaccctttttccactttgggttattttgggaacacttgttggcatcttcaagccatccttggttacatggttggaaacagatctctttactctaggtcttggatttcttatgctttccatgggtttgactcttacgtttgaagatttcagaagatgtttacgtaatccatggacggtgggtgttggttttcttgctcaatatatgatcaagccaattctaggttttctcattgcaatgactcttaagctttcggcacctcttgcgactggccttatcctagtctcatgctgccctggaggacaggcgtcaaacgttgctacttacatttccaaggggaatgtagcgctctctgtactcatgacaacgtgttcaaccattggggctattataatgactcctcttcttactaagcttcttgctggtcagcttgttcccgttgacgctgctggacttgctcttagtacgttccaagtagtgttggttcctaccataattggagttctggcaaatgagttctttcctaaatttacgtctaagatcataacagtgacgcctctaatcggagtcattctgactactctgctctgtgccagccctattggacaagttgcagatgttttgaaaacccaaggagctcaacttatactcccggtggcactccttcatgctgcagcctttgctattggctattggatttcaaagttttctttcggcgagtccacttcgcgtaccatttctatagaatgtggaatgcaaagttcagcgctcgggttcttgcttgcacaaaagcatttcacaaaccctctagttgctgttccttctgcagtcagtgttgtctgtatggcgcttggcgggagcggcctggccgtgttctggagaaacctaccgattccggcagatgacaaggatgacttcaaagagtaa

SEQ ID NO: 3 - DNA, 28 bp, Artificial Sequence
  AtBASS2_F1
gaattcatggcttccatttccagaatct

SEQ ID NO: 4 - DNA, 28 bp, Artificial Sequence
  AtBASS2_R1
ctcgagttactctttgaagtcatccttg

SEQ ID NO: 5 - DNA, 22 bp, Artificial Sequence
  AtBASS2_F2
aggtgacttacctgagagtact

SEQ ID NO: 6 - DNA, 21 bp, Artificial Sequence
  AtBASS2_R2
gtaagtagcaacgtttgacgc

(19) United States
(12) Patent Application Publication
(10) Pub. No.: US 2018/0094270 A1
(43) Pub. Date: Apr. 5, 2018

(54) COMPOSITION, CONTAINING BASS2 PROTEIN OR GENE ENCODING SAID PROTEIN, FOR INCREASING SIZE OF PLANT SEEDS AND CONTENT OF DEPOT FAT IN SEEDS

(71) Applicant: POSTECH ACADEMY-INDUSTRY FOUNDATION, Pohang-si (KR)

(72) Inventors: Youngsook LEE, Pohang-si (KR); Eun-Jung LEE, Gapyeong-gun (KR); Minwoo OH, Uijeongbu-si (KR)

(21) Appl. No.: 15/538,738

(22) PCT Filed: Sep. 7, 2015
(86) PCT No.: PCT/KR2015/009411

§ 371 Date: Jun. 22, 2017

(30) Foreign Application Priority Data
Dec. 26, 2014 (KR) 10-2014-0190188

(51) Int. Cl. C12N 15/82; A01H 5/10; C07K 14/415

(52) U.S. Cl. CPC C12N 15/8201; A01H 5/10; C12N 15/8247; C12N 15/8234; C07K 14/415

(57) ABSTRACT

The present invention relates to a technique of increasing the size of plant seeds and the content of storage fat in seeds by using a pyruvic acid transporter in charge of transporting pyruvic acid in a plant and, more specifically, to a composition for increasing the size of plant seeds and the content of storage fat in the seeds, the composition containing the BASS2 protein, which is a pyruvic acid transporter, or a gene encoding the protein, and to a method for increasing the size of plant seeds or the content of storage fat in the seeds, the method comprising a step of introducing the gene and a promoter for overexpressing the gene into a plant. According to the present invention, the fatty acid precursor can be increased by increasing the amount of pyruvic acid transported to the chromatophore at the time of forming seeds, thereby increasing the size of the seeds and the content of storage fat in the seeds, thus expecting the increase of productivity due to the increase in the seed yield. In addition, the content of storage fat in the seeds can be further increased through the increase in the size of the seeds, which corresponds to a main organ for storing plant fat, thereby significantly improving productivity of plant fat (oil) in a restricted space.

### TECHNICAL FIELD

The present invention relates to a composition for increasing the size of a plant seed and the content of storage fat in a seed using a BASS2 protein or a gene encoding the same, and a method therefor.

### BACKGROUND ART

Plants can directly produce energy sources through photosynthesis by absorbing water and carbon dioxide, and representative energy sources produced by plants are carbohydrates including sucrose, glucose, starch, etc., proteins and fat.

Among these, vegetable fat is expected to not only provide an essential energy source to a human, but also become a future bio-energy source which can replace fossil fuel, and therefore it is necessary to understand mechanisms of producing vegetable fat and regulating such production.

In addition, the demand for vegetable fat is getting higher, although supply is not keeping up with the demand. While due to continuous breeding and improved crossbreeding, current oil production from oilseed has reached the maximum, it is predicted that, according to the current breeding and crossbreeding methods, oil production in a limited cultivation area will not catch up with the demand. In recent years, a genetically-modified organism (GMO) has emerged to overcome such limitation. To produce vegetable fat which is expected to have huge demand worldwide, it is predicted that the development of GMOs with increased oil production is essential. In this regard, many scientists are conducting research to raise an oil content per seed unit weight and total production, but these goals are not easily achieved since various genes are intricately involved in the synthesis of triacylglycerols that account for most of the vegetable storage oil in seeds and the regulation of the synthesis.

To improve the storage oil in plant seeds, giant multinational corporations, for example, Monsanto, DuPont, etc. have conducted research. However, even when the storage fat in a seed increases, due to decreases in the total growth of the plant and the number of seed pods, overall productivity tends to decrease, and there are no reports of great performance yet. It is necessary to discover genes that can increase storage oil in seeds storing most of the vegetable fat, but have no change in size of the seed or the number of pods, or more ideally, have increases in both the seed size and the number of pods.

Meanwhile, during fat production in plants, pyruvate is important as an intermediate. In plant, pyruvate serves as a precursor for synthesis of fatty acids and a secondary metabolite as well as amino acid metabolism and energy production by the transport of the pyruvate from the cytoplasm to the plastid. In such transport of pyruvate from the cytoplasm to the plastid, a transport protein similar to the human bile acid sodium symporter (BASS) protein is involved, and Arabidopsis thaliana has 6 genes encoding a protein similar thereto. Among these, particularly, bile acid sodium symporter family protein 2 (BASS2) is located in the plastid of a leaf, and known to directly act on the pyruvate transport (Furumoto et al., Nature, 2011). The pyruvate transported into the plastid is converted into acetyl-coA, and then converted into malonyl-coA. The produced acetyl-coA and malonyl-coA are bound with two carbons by the enzymatic action of a fatty acid synthase complex, resulting in a 16:0-acyl carrier protein (ACP), 18:0-ACP and 18:1-ACP. Afterward, the resulting proteins are transported to the endoplasmic reticulum by the ATP binding cassette transporter A subfamily 9 (ABCA9) protein, and then participate in the synthesis of phospholipids which constitute a cell membrane and triacylglycerols (TAG) which are storage fats, through the modification and combination of fatty acids.

The family Brassicaceae, including Arabidopsis thaliana, is a family of plants that store fat in seeds, and the fat accounts for approximately 37% of a seed weight. Since the fat in the plant seed can store lots of energy as well as serving as an energy source, the fat is a very important material associated with the production of bio-energy. Accordingly, when sink strength is enhanced by increasing the transport of a precursor used in the synthesis of fatty acids, it is expected that the amount of fat which is synthesized and then stored in seeds can be greatly increased.

Therefore, to date, while many studies have been conducted to increase the fat content in seeds, the studies mainly focus on a fatty acid synthase, a synthase of a triacylglycerol which is storage neutral fat, and the overexpression of transcriptional regulatory factors for these proteins, but there is little known about research using fat and a fat precursor transporter.

### DISCLOSURE

### Technical Problem

To increase the content of fat in plant seeds, the present invention is directed to providing a technique of increasing the size of a plant seed and/or the content of storage fat in a seed using a pyruvate transporter BASS2 protein or a gene encoding the same.

However, technical problems to be solved in the present invention are not limited to the above-described problems, and other problems which are not described herein will be fully understood by those of ordinary skill in the art from the following descriptions.

### Technical Solution

To achieve the object of the present invention, the present invention provides a composition for increasing the size of a plant seed and the content of storage fat in a seed, comprising one or more selected from the group consisting of a bile acid:sodium symporter 2 (BASS2) protein of a plant and a gene encoding the same.

In one exemplary embodiment of the present invention, the composition may include an expression vector for overexpressing the gene or a microorganism transformed with the expression vector.

In addition, the present invention provides a method for increasing the size of a plant seed and the content of storage fat in a seed, which includes introducing an expression vector including a gene encoding the BASS2 protein of a plant into a plant body.

In one exemplary embodiment of the present invention, the BASS2 protein may be a polypeptide consisting of an amino acid sequence of SEQ ID NO: 1.

In one exemplary embodiment of the present invention, the expression vector may include a promoter for overexpressing the gene.

In one exemplary embodiment of the present invention, the storage fat in a seed may be a triacylglycerol.

In one exemplary embodiment of the present invention, the plant may be selected from the group consisting of cabbage, radish, broccoli, Brassica juncea, Arabidopsis thaliana, rapeseed, camelina, sunflower, flax, cotton, soybean, safflower, canola, sesame, perilla, peanut, castor-oil plant, calendula, rose, coconut, palm tree, grape, apricot, rice, corn, grass, microalgae, and plum.

In addition, the present invention provides a plant body which is increased in seed size and the content of storage fat in a seed according to the method.

In one exemplary embodiment of the present invention, the plant body may be selected from the group consisting of tissue, a cell and a seed of a plant.

In addition, the present invention provides a seed which is increased in size and the content of storage fat by performing the method.

Further, the present invention provides a use of the composition to increase the size of a plant seed and the content of storage fat in a seed.

### Advantageous Effects

The present invention can cause an increase of a fatty acid precursor by increasing the amount of pyruvate transported to the plastid in seed formation by overexpressing a BASS2 protein as a transporter serving to transport pyruvate in the plastid of a plant or a gene encoding the same in a developing seed and a structure for protecting a seed, and ultimately can increase a seed size and the amount of storage fat in a seed.

In addition, as the size of a plant seed and the content of storage fat in a seed are increased, productivity can be expected to increase due to an increased fat yield, and the content in storage fat in a seed may be further increased by increasing the size of a seed, which is a main organ for storing vegetable fat, and thus the productivity of vegetable fat (oil) can be considerably increased in a restricted space.

### DESCRIPTION OF DRAWINGS

FIG. 1 is the cleavage map of a vector in which a CDS region of a pyruvate transporter BASS2 is inserted behind the glycinin promoter of pBinGlyBar1.

FIG. 2 shows a real-time polymerase chain reaction (real-time PCR) result demonstrating that BASS2 transcription is greatly increased in a developing silique of a plant in which a pyruvate transporter BASS2 is overexpressed.

FIG. 3 shows the result of measuring the size of a seed of a pyruvate transporter BASS2-overexpressing transformant, compared with that of a wild type.

FIG. 4 shows the result of measuring the total fat content in a pyruvate transporter BASS2-overexpressing seed using a fatty acid methyl ester (FAME), compared with that of a wild type.

FIG. 5 shows the result of measuring an amount of C20:1, which is a representative neutral fat, in the total content of fat extracted from a pyruvate transporter BASS2-overexpressing transformant, compared with a wild type.

FIG. 6 shows the composition of a fatty acid of the total fat analyzed from a pyruvate transporter BASS2-overexpressing seed, which is represented in percentage.

FIG. 7 shows amounts of a protein, starch and sucrose extracted from a pyruvate transporter BASS2-overexpressing seed, compared with a wild type.

FIG. 8A shows comparison in the number of siliques measured from the main stem between a wild type and a pyruvate transporter BASS2-overexpressing transformant after sowing.

FIG. 8B shows the number of seeds present per silique for the central stem of a pyruvate transporter BASS2-overexpressing transformant after sowing, compared with a wild type.

FIG. 8C shows comparison in the number of seeds per single plant body between a wild type and a pyruvate transporter BASS2-overexpressing transformant after sowing.

### MODES OF THE INVENTION

The inventors confirmed that the size of a plant seed and the content of storage fat in a seed are increased when an increase in the pyruvate transport to the plastid is induced by overexpressing a pyruvate transporter BASS2 (bile acid:sodium symporter 2) protein serving to transport pyruvate to the plastid of a plant, contributing to fat synthesis in seed development of a plant, and thus completed the present invention.

Therefore, the present invention provides a composition for increasing the size of a plant seed and the content of storage fat in a seed, comprising BASS2 protein or a gene encoding the same.

In the present invention, the BASS2 protein is a polypeptide consisting of an amino acid sequence represented by SEQ ID NO: 1, and includes a functional equivalent of the protein. The term “functional equivalent” is a protein having at least 70% or more, preferably 80% or more, more preferably 90% or more, and further more preferably 95% or more sequence homology with the amino acid sequence represented by SEQ ID NO: 1 as a result of the addition, substitution or deletion of amino acids, and exhibiting substantially the same physiological activity as the protein represented by SEQ ID NO: 1. The term “substantially the same physiological activity” refers to the activity of increasing the size of a plant seed and the content of storage fat in a seed in a plant body.

In addition, in the present invention, the gene encoding the BASS2 protein includes both genomic DNA encoding the BASS2 protein and cDNA thereof. Preferably, the gene may be a BASS2 CDS sequence represented by SEQ ID NO: 2, and a variant of the sequence is included in the scope of the present invention. In detail, the gene may include a base sequence having 70% or more, more preferably 80% or more, further more preferably 90% or more, and most preferably 95% or more sequence homology with the base sequence of SEQ ID NO: 2. The “percent (%) sequence homology” with respect to the polynucleotide is determined by comparing comparative regions with two optimally aligned sequences, and a part of a polynucleotide sequence in the comparative region may include additions or deletions (that is, gaps), compared with a reference sequence (not including additions or deletions) with respect to the optimal alignments of the two sequences.

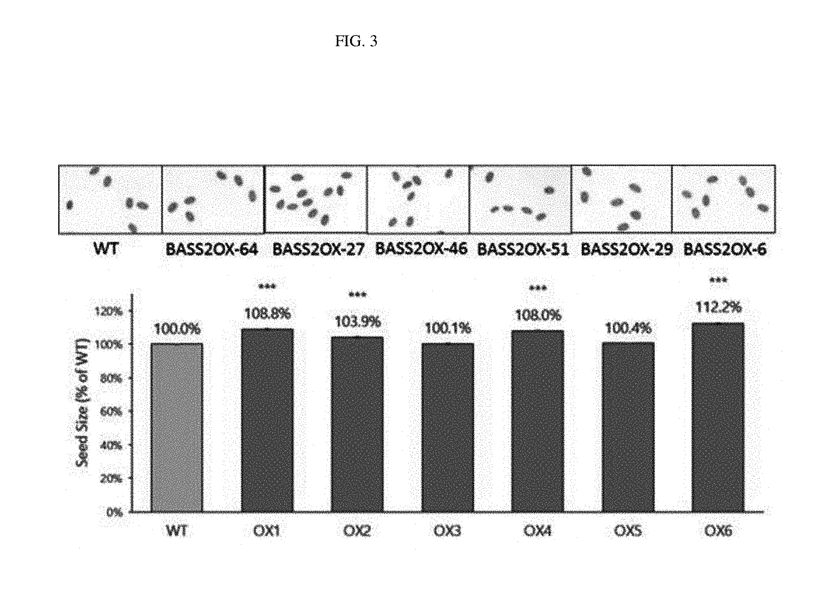

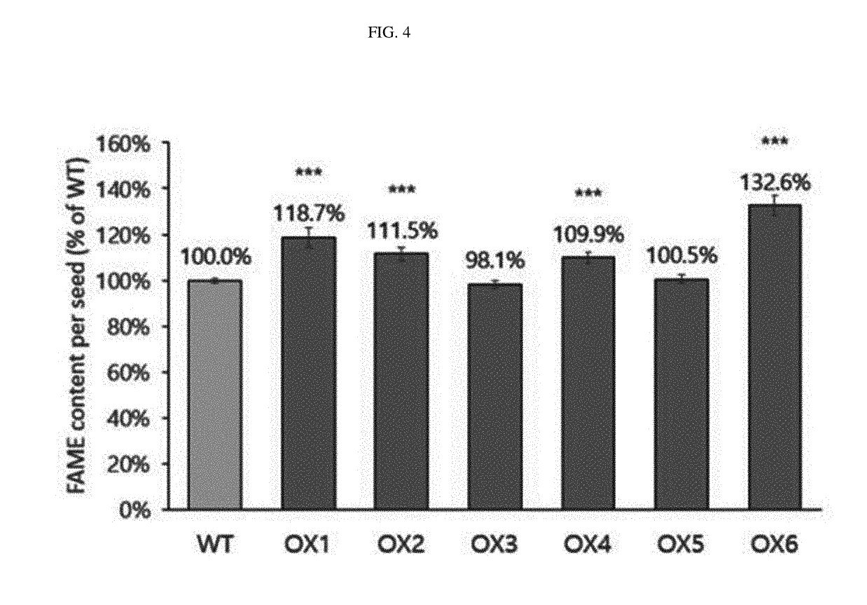

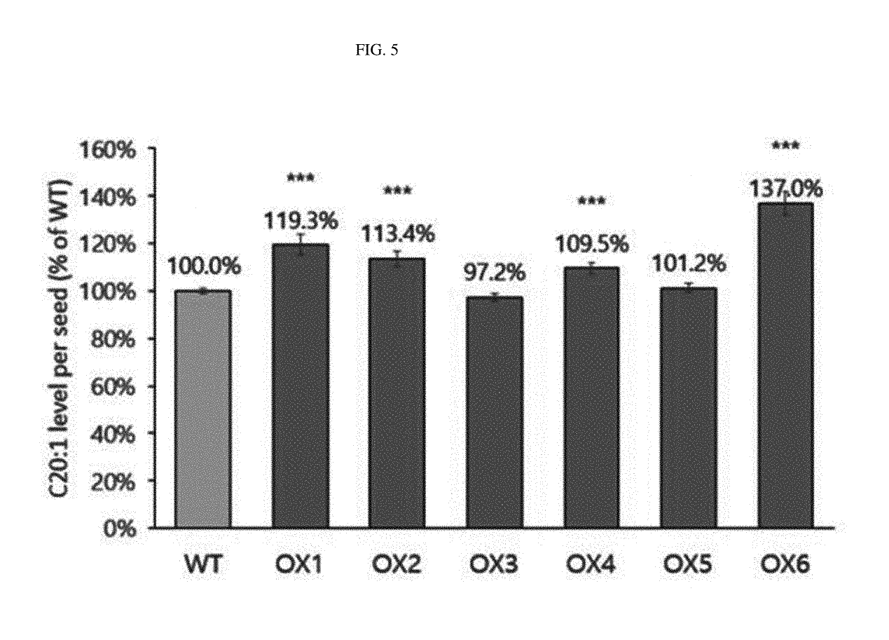

The composition according to the present invention increases the size of a plant seed and the content of storage fat in the seed, and in one exemplary embodiment of the present invention, it was confirmed that a BASS2-overexpressing transgenic plant (transformant) shows a phenotype with an increased seed size, compared with a wild type (refer to FIG. 3), and as a result of the confirmation of fat contents, it was confirmed that the total fat content is considerably increased, compared with the wild type (refer to FIG. 4), and particularly, the content of storage fat, triacylglycerols, is remarkably increased (refer to FIG. 5).

From the above results, the present invention demonstrated that BASS2 overexpression in a plant leads to an increase in pyruvate transport to the plastid from the cytoplasm, resulting in increases in a seed size and the content of storage fat in a seed, and thus the present invention may provide a composition including one or more selected from the group consisting of a BASS2 protein of a plant and a gene encoding the same to increase the size of a plant seed and the content of storage fat in a seed.

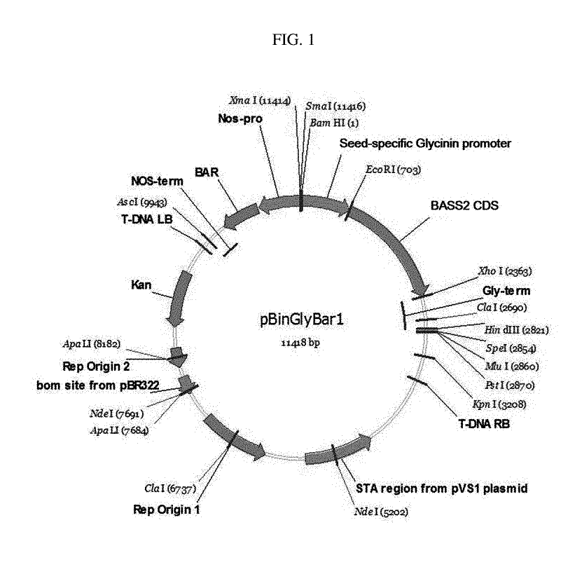

In one exemplary embodiment of the present invention, a BASS2-overexpressing transgenic plant body was manufactured using a soybean (bean) promoter (refer to FIG. 1). Therefore, the composition according to the present invention provides a transformation vector into which a gene encoding the BASS2 protein and a promoter for overexpressing the gene are inserted, or a plant transformed with the transformation vector.

In the present invention, the term “overexpression” refers to the expression of the BASS2 protein or gene encoding the same of the present invention over a level expressed in a wild type plant, and an overexpression method is not particularly limited, but may be performed using various known techniques. For example, the overexpression method may be performed by increasing a copy number of a suitable gene through mutation or introduction of a ribosome-binding site or promoter and a regulatory region, which are located upstream from a structural gene, and an expression cassette introduced upstream of the structural gene may act in the same manner. In addition, an inducible promoter of the gene encoding the BASS2 protein of the present invention may increase expression, and the expression may also be increased by a method for elongating the lifetime of mRNA. Further, the gene may be overexpressed by changing the composition of a medium and/or a culture technique.

Here, the term “transformation” refers to a molecular biological technique in which a DNA chain fragment or plasmid having a different type of foreign gene from that of the cell, which penetrates between cells to be bound with DNA originally present in the cell, thereby changing the genetic character of the cell. In the present invention, the transformation refers to the insertion of the gene encoding the BASS2 protein into a plant, along with the overexpression promoter.

In addition, the term “transformation vector” refers to a recombinant DNA molecule including a suitable nucleic acid sequence required for expressing a target coding sequence, and a coding sequence operably linked in a specific host organism. The suitable nucleic acid sequence may be a promoter, and may further include an enhancer, a transcription terminator and a polyadenylation signal. Promoters, enhancers, transcription terminators and polyadenylation signals, which are able to be used in eukaryotes, are known. The transformation vector may be a plant expression vector which may be directly introduced into a plant cell by inserting the base sequence of the gene, or may be introduced into a microorganism causing infection in a plant. An exemplary example of the transformation vector is a Ti-plasmid vector which may transfer a part of the vector itself, that is, a T-region, when present in a suitable host such as Agrobacterium tumefaciens, to a plant cell. There are various Agrobacterium strains, which can be used in such manipulation, and are known in the art. Currently, different types of Ti-plasmid vectors are used to transfer a hybrid DNA sequence to a plant cell, or a protoplast capable of producing a new plant by suitable insertion of hybrid DNA into a plant genome. A particularly exemplary type of the Ti-plasmid vector is a binary vector. A different vector suitable for introducing the DNA according to the present invention into a plant host may be a viral vector which may be derived from a double-stranded plant virus (e.g., CaMV) and a single-stranded virus, a geminivirus, or the like, such as an incomplete plant viral vector. The vector may be advantageously used when it is difficult to suitably transform a plant host. Preferably, the transformation vector may further include a marker capable of identifying the expression of the gene or selecting a transformant. The marker is a nucleic acid sequence characterized by being conventionally selected by a chemical method, and includes all genes that can differentiate transformed cells from non-transformed cells. As a marker gene, a gene exhibiting resistance against antibiotics such as kanamycin, spectinomycin, etc. or a gene encoding β-glucuronidase (GUS) or a green fluorescence protein (GFP) may be used, but the present invention is not limited thereto. The marker is transferred to a plant, together with the vector, and cultured in a medium containing a specific antibiotic to enable the selection of a transformant.

In addition, the “promoter” is a promoter for plant expression, and may include, but is not limited to, the cauliflower mosaic virus (CaMV) 35S promoter, the nopaline synthase (NOS) promoter of the Agrobacterium tumefaciens Ti plasmid, the octopine synthase (OCS) promoter, or the mannopine synthase (MAS) promoter as well as other known promoters.

In addition, here, the microorganism may be used without limitation as long as it causes infection in a plant.

In addition, the present invention provides a method for increasing the size of a plant seed or the content of storage fat in a seed, which includes introducing an expression vector including a gene encoding a BASS2 protein of a plant into a plant body.

Here, the gene may be inserted into an expression vector, and a method for transforming the gene-inserted expression vector into a plant body may be an Agrobacterium tumefaciens-mediated DNA transfer method, and preferably, a method for immersing recombinant Agrobacterium prepared using electroporation, micro-particle injection or a gene gun. However, the present invention is not limited thereto, and various known methods may be used.

In the present invention, the “increase in the size of a plant seed” may include all of increases in the weight and volume of a seed, the number of pods, the size of pods and the resulting increases in yield and productivity of seeds as well as the increase in the size of a plant seed, but the present invention is not limited thereto. In addition, the “increase in the content of storage fat in a seed” refers to an increase in the content of fat (or oil) stored in a plant seed, and such an increase in the content of storage fat in a seed may include, but is not limited to, both an increase in the content of storage fat in a seed and an increase in the productivity of vegetable fat (or oil) caused thereby, which are caused by the above-described increase in seed size, as well as an increase in the content of storage fat itself. A representative example of the storage fat in a seed is a triacylglycerol. Triacylglycerols (a triacylglycerol; a triacylglyceride; a triglyceride) are structures including three fatty acids binding to one glycerol as a backbone, and main components in vegetable fat and animal fat. Such triacylglycerols are representative storage fats which are converted for use as an energy source in the case of the lack of carbohydrates in animals, and in the case of a plant, generally stored in a seed and used as a nutrient in germination.

In the present invention, plants may be all types of crops or flowers requiring increases in the size of a plant seed and the content of storage fat in a seed, and may include, but are not limited to, cabbage, radish, broccoli, Brassica juncea, Arabidopsis thaliana, rapeseed, camelina, sunflower, flax, cotton, soybean, safflower, canola, sesame, perilla, peanut, castor-oil plant, calendula, rose, coconut, palm tree, grape, apricot, rice, corn, grass, microalgae, and plum. However, the present invention demonstrates that an increase in the BASS2 protein in a plant results in the increases in the size of a plant seed and the content of storage fat in a seed, and it is apparent to those of ordinary skill in the art that an applicable plant body is not limited to any one of these examples.

Further, the present invention provides a plant body increased in a seed size or the content of storage fat in a seed according to a method for increasing the size of a plant seed or the content of storage fat in a seed, and a seed increased in size or storage fat content by performing the above-described method.

The plant body may be tissue, a cell or a seed of a plant, but the present invention is not limited thereto. The “tissue of a plant” includes tissue of a differentiated or non-differentiated plant, such as a root, a stem, a leaf, pollen, a seed, cancerous tissue, and various types of cells used for culture, that is, single cells, protoplasts, buds and callus tissue, but the present invention is not limited thereto. The tissue may be in planta or in an organ culture, tissue culture or cell culture. In addition, the “plant cell” may be any plant cell, and preferably a cultured cell, a cultured tissue, a cultured organ or an entire plant, and can be any type without limitation.

Hereinafter, to assist the understanding of the present invention, exemplary examples will be provided. However, the following examples are merely provided to more easily understand the present invention, and the scope of the present invention is not limited to the following examples.

### Experimental Example

In the present invention, RNA isolation, cDNA synthesis and PCR were performed under conditions and by methods as follows. First, a sample was quickly cooled using liquid nitrogen, and evenly homogenized. To the homogenized sample, 900 μl of TRIzol was added and sufficiently mixed, and then the resulting mixture was maintained at 65° C. for 10 minutes and mixed with 200 μl of chloroform. Afterward, the obtained mixture was centrifuged at 12000 rpm and 4° C. for 15 minutes, and then a supernatant was transferred to a new tube. Here, 600 μl of isopropanol was added to the tube, and the tube was maintained at room temperature for 10 minutes, centrifuged at 12000 rpm and 4° C. for 10 minutes, and then a supernatant was removed therefrom. Afterward, pellets were washed with 500 μl of 75% ethanol and centrifuged again at 12000 rpm and 4° C. for 10 minutes, and then a supernatant was removed. The remaining pellets were treated at 65° C. for 5 minutes to completely remove ethanol, and treated with DNase I for 30 minutes to remove DNA. A reaction was then carried out at 75° C. for 10 minutes to inactivate DNase I, and RNA obtained thereby was used for cDNA synthesis.

To synthesize cDNA using the obtained RNA, cDNA was synthesized according to a manufacturer's method using GoScript reverse transcriptase (Promega) and an oligo dT primer. Polymerase chain reaction (PCR) and real-time PCR were performed using the synthesized cDNA as a template (94° C. for 3 minutes and 94° C. for 5 seconds, 56° C. for 15 seconds, 72° C. for 30 seconds, 45 cycles, 95° C. for 15 seconds, 60° C. for 30 seconds, and 95° C. for 15 seconds). The UBIQUITIN11 (UBQ11) gene was used as a normalization control for relatively comparing the amount of total cDNA used per sample.

In the present invention, lipid extraction and isolation/quantification of neutral fat were performed under conditions by a method as follows. First, 30 seeds were placed in a glass tube, and then 50 nmol of C17:0 triacylglycerol was added to be used as a standard in quantitative comparison. Here, 1 ml of a 5% sulfuric acid/methanol solution and 300 μl of toluene were added and mixed for 30 seconds. Afterward, the resulting solution was cooled at 90° C. for 90 minutes. Here, the resulting solution was mixed with 1.5 ml of a 0.9% potassium hydroxide solution and 2.5 ml of hexane by shaking and centrifuged at 1500 rpm for 5 minutes, and then a supernatant was transferred to a new tube. The supernatant was evaporated using a nitrogen gas, remaining pellets were defrosted with 5 drops of hexane, and then the resulting solution was analyzed by gas chromatography-mass spectrophotometry.

### EXAMPLES

### Example 1. Design/Discovery of Plant for Overexpressing Pyruvate Transporter BASS2 in Developing Silique

Pyruvate is produced in the cytoplasm through glycolysis, is transported to the plastid, and is thus used as a precursor for synthesis of isoprene and fatty acids. Accordingly, to investigate if an increase in pyruvate transport to the plastid contributes to fat synthesis during seed development, the inventors designed a vector capable of expressing a gene encoding the pyruvate transporter, BASS2, in a developing silique and a seed of Arabidopsis thaliana. To this end, RNA was extracted from an Arabidopsis thaliana plant to synthesize cDNA, and PCR was performed using a forward primer AtBASS2_F1 (SEQ ID NO: 3: 5′-GAATTCATGGCTTCCATTTCCAGAATCT-3′) and a reverse primer AtBASS2_R1 (SEQ ID NO: 4: 5′-CTCGAGTTACTCTTTGAAGTCATCCTTG-3′), which are capable of specifically binding to AtBASS2 cDNA. As shown in FIG. 1, a CDS region of the gene of the synthesized pyruvate transporter BASS2 was introduced behind the soybean glycinin promoter in the pBinGlyBar1 vector. In addition, the designed vector was introduced to an Arabidopsis thaliana wild type, thereby manufacturing a BASS2 transformant line, and then the line was selected.

### Example 2. Analysis of BASS2 Overexpression Pattern of Transformed Plant

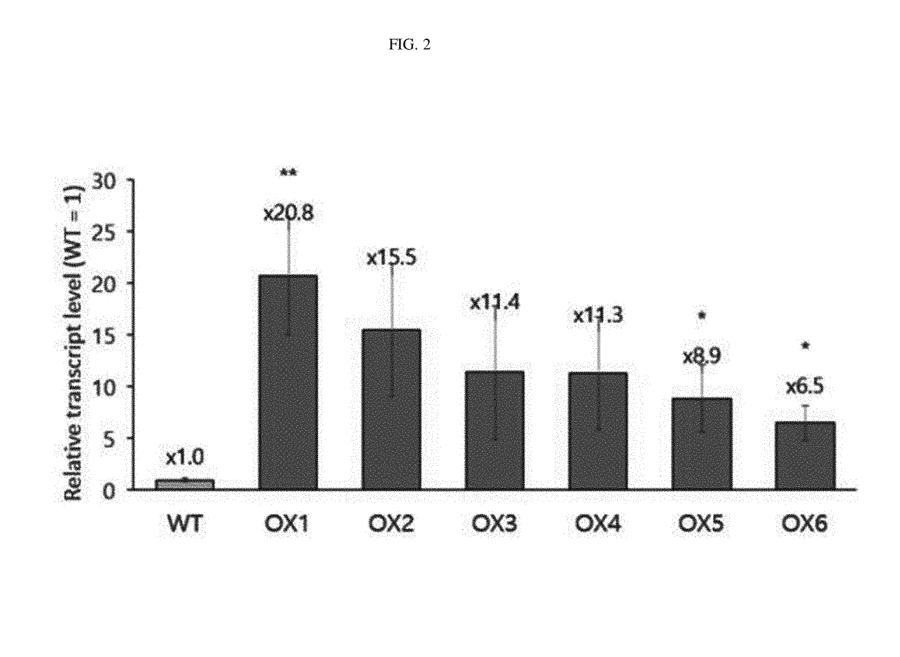

BASS2 overexpression in a developing silique and seed of the pyruvate transporter BASS2 transformant line manufactured in Example 1 was examined. To this end, developing siliques and seeds were harvested from an earth-grown wild type and a BASS2 transformant plant on DAF 12 to 14, and RNA was extracted therefrom to synthesize cDNA, and then quantitative real-time PCR was performed using a forward primer AtBASS2_F2 (SEQ ID NO: 5: 5′-AGGTGACTTACCTGAGAGTACT-3′) and a reverse primer AtBASS2_R2 (SEQ ID NO: 6: 5′-GTAAGTAGCAACGTTTGACGC-3′), which are capable of specifically binding to AtBASS2 cDNA, using the cDNA as a template (conditions: 94° C. for 3 minutes, [94° C. for 5 seconds, 56° C. for 15 seconds, 72° C. for 30 seconds]*45 cycles, 95° C. for 15 seconds, 60° C. for 30 seconds, 95° C. for 15 seconds). As a result, as shown in FIG. 2, it was confirmed that, in the BASS2 transformant, the level of transcription was considerably higher than that in the wild type.

This result indicates that BASS2 expression was considerably increased in developing siliques and seeds of these transformants.

### Example 3. Analysis of Seed Size of Pyruvate Transporter BASS2-Overexpressing Transformant

To examine whether the overexpression (OX) of the pyruvate transporter BASS2 induces differences in a seed size and the fat content during seed formation, seeds were harvested from the plant body of FIG. 2 and photographed, and cross-sectional areas of seeds were measured using an imaging program (Image J) and then compared with the wild type. As a result, as shown in FIG. 3, it was confirmed that, in four out of six BASS2 transformants, a seed size was larger than that of the wild type. It was confirmed that, although such phenotypes had a difference in size increment of seeds according to BASS2 transformant lines, the seed size was increased up to approximately 103% to 112%.

From the above result, it was confirmed that the phenotype in which the seed size of the BASS2-overexpressing transformant was increased was caused by the introduction of the pyruvate transporter BASS2.

### Example 4. Analyses of Total Lipid and Content and Composition of Storage Fat in Seed of Pyruvate Transporter BASS2-Overexpressing Transformant

To examine whether the increase in seed size is caused by the increase in fat content in the BASS2-overexpressing transgenic plant body identified in Example 3, lipid in a seed was extracted, and its content was analyzed. To this end, all of lipid products present in a seed were degraded into a fatty acid methyl ester (FAME) using a sulfuric acid/methanol solution, and FAME was dissolved in hexane and then used for quantification and analysis of the composition of a fatty acid using GC-MS. As a result, as shown in FIG. 4, it can be seen that a total lipid content present in seeds was considerably increased in the BASS2-overexpressing transformant lines showing the increased seed size. This result indicates that the pyruvate transporter BASS2-overexpressing transformants tend to show similar increases in seed size and fat content.

Subsequently, for analysis of the content of storage fat, a triacylglycerol, of the total fat in the seed, an amount of the representative fatty acid C20:1 was measured and compared with a wild type. As shown in FIG. 5, it was confirmed that in all of the pyruvate transporter BASS2-overexpressing transformant lines showing the increases in seed size and total fat content, the amount of C20:1 was considerably increased, compared to that in the wild type.

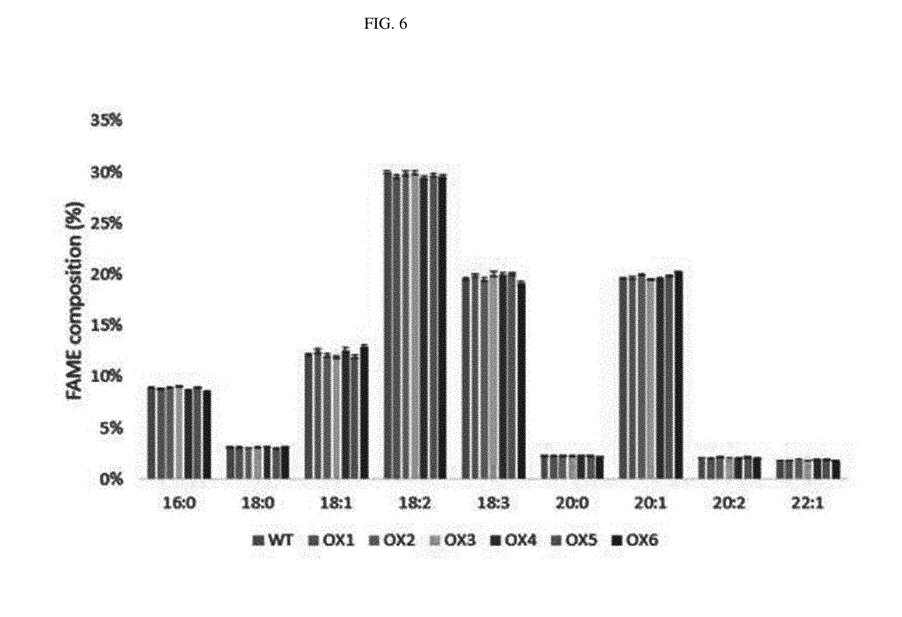

In addition, as a result of analysis of the composition of fatty acids extracted from a seed of the pyruvate transporter BASS2-transformant, as shown in FIG. 6, it can be seen that the ratio of all fatty acids is similar to that of the wild type.

From this result, it can be seen that contents of all fatty acids are increased in a seed of the BASS2-overexpressing transformant, and therefore an accumulative amount of the storage fat, a triacylglycerol, of the seed was also increased.

### Example 5. Analyses of Contents of Protein and Carbohydrate in Seed of Pyruvate Transporter BASS2-Overexpressing Transformant

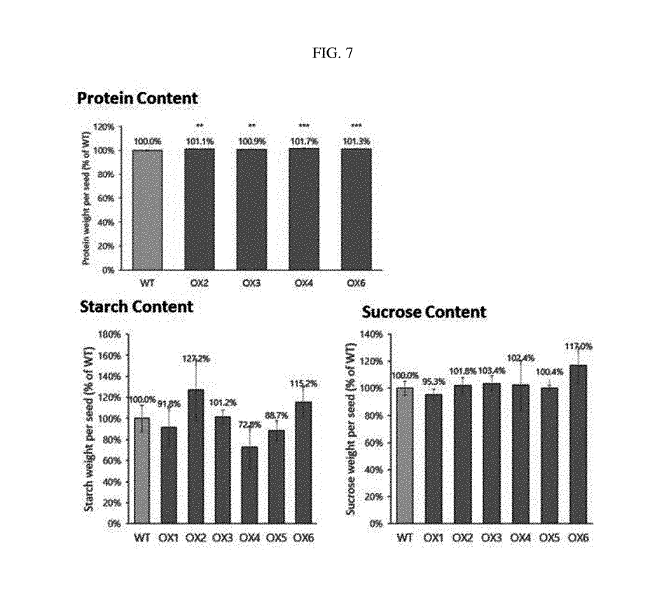

To examine whether the increase in seed size in the BASS2-overexpressing transgenic plant body identified in Example 3 is caused by the increases in contents of protein and carbohydrate, which are other seed metabolites, as well as a fat content, protein, sucrose and starch of a seed were extracted to analyze their contents. As a result, as shown in FIG. 7, it can be seen that amounts of the protein, sucrose and starch present in seeds in the BASS2-overexpressing transformant lines showing the increased seed size were slightly decreased or increased, but were not much different from those of the wild type.

From this result, it can be seen that the increase in seed size of the BASS2-overexpressing transformant is not caused by the increase in protein or carbohydrate, but by the increase in fat content, compared to the wild type.

### Example 6. Analysis of Seed Yield of Pyruvate Transporter BASS2-Overexpressing Transformant

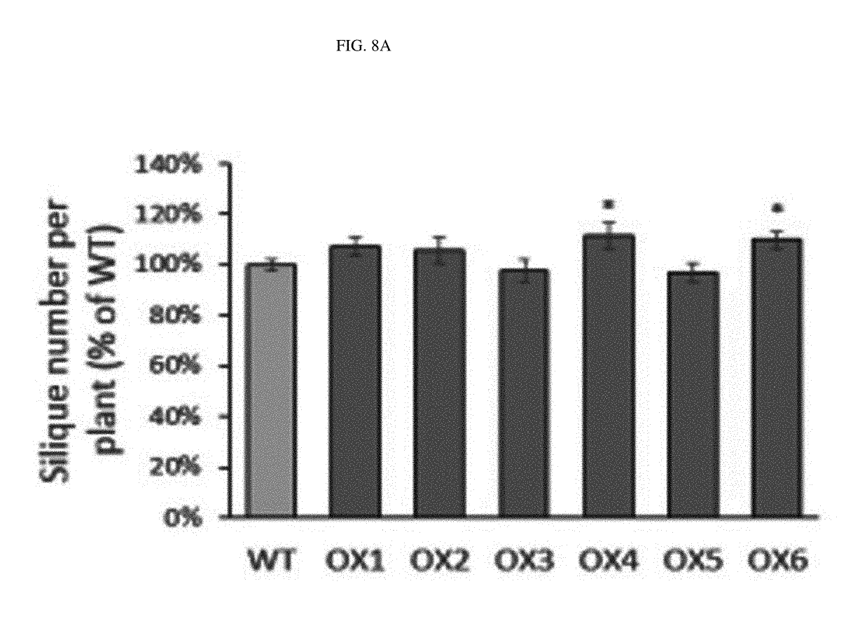

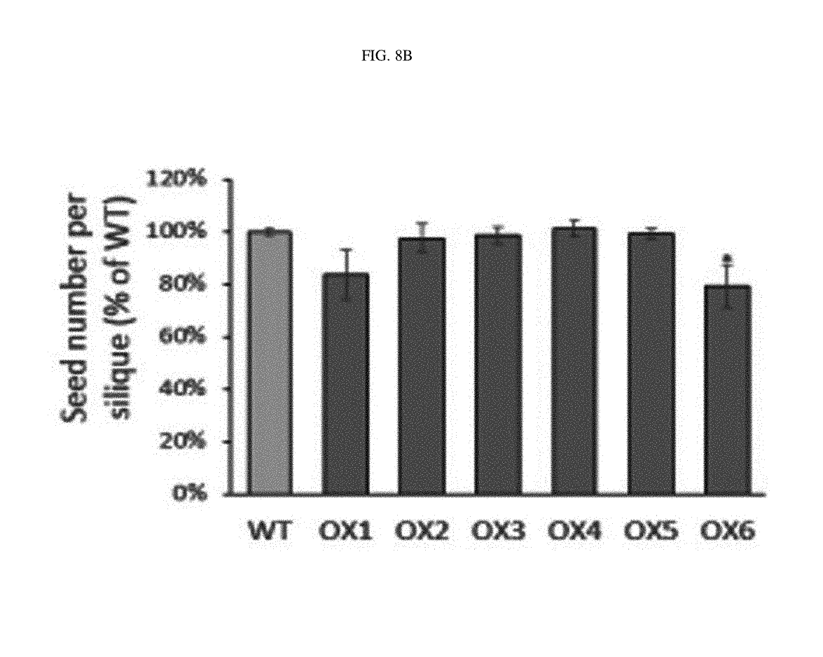

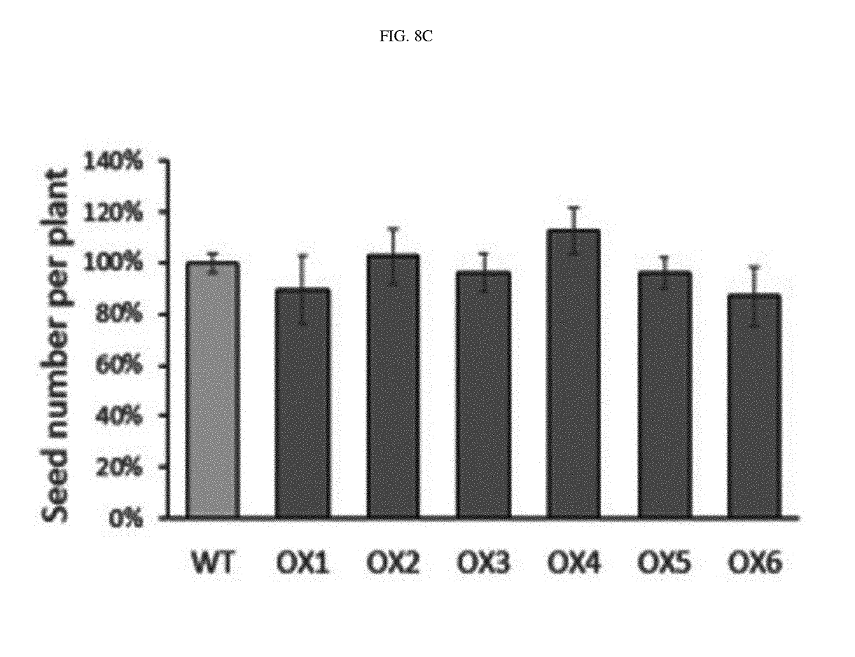

Due to the increases in seed size and fat content in a single seed, there is a chance of a decreased seed yield from a single plant body, and therefore, to confirm this, the number of siliques grown on the main stem and the number of seeds present in a silique of a pyruvate transporter BASS2-overexpressing transformant were observed. As a result, as shown in FIGS. 8A and 8B, in all lines except OX4 and OX6, the number of siliques and the number of seeds were similar to those of the wild type. In addition, as a result of calculation of seed yield in a single plant body, shown in FIG. 8C, it was confirmed that the seed yield was not different from that of the wild type.

From this result, it can be seen that the phenotype shown in the BASS2-overexpressing transformant does not influence the seed yield of a single plant.

It would be understood by those of ordinary skill in the art that the above descriptions of the present invention are exemplary, and the exemplary embodiments disclosed herein can be easily modified into other specific forms without changing the technical spirit or essential features of the present invention. Therefore, it should be interpreted that the exemplary embodiments described above are illustrative in all aspects and not limiting.

1-5. (canceled)

6. A method for increasing the size of a plant seed and the content of storage fat in a seed, comprising:
introducing an expression vector including a gene encoding a bile acid:sodium symporter 2 (BASS2) protein of a plant into a plant body.

7. The method of claim 6, wherein the BASS2 protein is a polypeptide consisting of an amino acid sequence of SEQ ID NO: 1.

8. The method of claim 6, wherein the expression vector includes a promoter for overexpressing the gene.

9. The method of claim 6, wherein the storage fat in a seed is a triacylglycerol.

10. The method according to claim 6, wherein the plant is selected from the group consisting of cabbage, radish, broccoli, Brassica juncea, Arabidopsis thaliana, rapeseed, camelina, sunflower, flax, cotton, soybean, safflower, canola, sesame, perilla, peanut, castor-oil plant, calendula, rose, coconut, palm tree, grape, apricot, rice, corn, grass, microalgae, and plum.

11. A plant body which is increased in seed size and the content of storage fat in a seed according to the method of claim 6.

12. The plant body of claim 11, which is selected from the group consisting of tissue, a cell and a seed of a plant.

13. A seed which is increased in size and the content of storage fat by performing the method of claim 6.

14-17. (canceled)
